(19) United States
(12) Patent Application Publication
(10) Pub. No.: US 2022/0039664 A1
(43) Pub. Date: Feb. 10, 2022

(54) Body temperature display device

(71) Applicants: Bradford Brian Hutson, SR., Vero Beach, FL (US); Bradford Brian Hutson, JR., Green Cove Springs, FL (US); Hoyt Mac Layson, JR., Orlando, FL (US)

(72) Inventors: Bradford Brian Hutson, SR., Vero Beach, FL (US); Bradford Brian Hutson, JR., Green Cove Springs, FL (US); Hoyt Mac Layson, JR., Orlando, FL (US)

(21) Appl. No.: 17/300,486

(22) Filed: Jul. 19, 2021

Related U.S. Application Data
(60) Provisional application No. 63/103,161, filed on Jul. 22, 2020.

(51) Int. Cl. A61B 5/01; A61B 5/00

(52) U.S. Cl. CPC A61B 5/01; A61B 2560/0214; A61B 5/6803; A61B 5/742

(57) ABSTRACT

The apparatus disclosed in this Patent displays the wearer's body temperature as normal, fever, or unknown using multicolor LEDs (Light Emitting Diodes). The apparatus can be body worn in multiple form factors such as PPE (Personal Protective Equipment), clothing accessories, or transdermal patches.

### TECHNICAL FIELD

This disclosure relates to a body worn body temperature display device that will display the body temperature of the wearer using a multicolor Light Emitting Diode (LED). The color if the LED indicates if wearer's body temperature is within or above normal range. More specifically this disclosure relates to a LED display on the body temperature display device that will provide indication of normal or abnormal body temperature of the wearer and that indicates the health and status of the body temperature display device

### BACKGROUND OF THE INVENTION

Elevated body temperature is an early indicator for the presence of bacterial or viral infection. For contagious infections, elevated body temperature is used as a screening method for people that are not or are yet exhibiting other more obvious symptoms such as coughing, sneezing, difficulty breathing, joint pain, diarrhea, and vomiting.

Body temperature may be required for admission or entry into:
Social gathering environments such as sporting events, restaurants, clubs, bars, amusement parks, etc.
Workplace environments,
School environments,
Mass transit environments

It has become important regarding pandemics to be able to rapidly screen people for temperature who are otherwise asymptomatic.

Manual screening of body temperature using contacting or non-contacting temperature probes requires personnel to operate the probe and can be a pedestrian traffic choke point because of data acquisition time especially for entry points that deal with rapid ingress requirements and large numbers of people.

Ideally, a body temperature display device that can be body worn when fitted to the exposed skin minutes prior to admission or entry, would allow for body temperature display device to acclimate to the body temperature of the wearer. Upon temperature stabilization, the body temperature display device would serve to expedite flow through ingress and egress points.

For extended stay environments, then continuous body temperature display would rapidly identify people for further examination, isolation, or quarantine.

Ideally the body worn body temperature display device should be easily applied to exposed skin or inserted into body worn articles that could be affixed to exposed skin, fit snugly to the exposed skin of the wearer, be cost effective, have long battery life, be water proof, be hypoallergenic to prevent allergic reaction during skin contact, and be disposable upon battery failure.

### SUMMARY OF THE INVENTION

According to the present state of the art, it is therefore the object of this invention to provided a low cost, battery operated body temperature display device with a simple body temperature normal or abnormal multicolor LED indicator, that is secured to the exposed skin of the wearer by a compression fit such as elastic bands, adjustable straps, apparel, face masks, face shields, or skin transdermal patches. Such a device would incorporate a medical certified temperature component integrated with the body temperature display device multicolor LED and battery which could either be permanent, replaceable or rechargeable. The multicolor LED color would indicate the status of the bracelet and the body temperature of the wearer. The multicolor LED color flashing orange when attached to the skin of the wearer would indicate that the body temperature display device status is acquiring the body temperature of the wearer. The multicolor LED color flashing orange would also indicate that the body temperature display device does not have proper contact with the skin of the wearer. The multicolor LED color green would indicate that the wearer's body temperature is normal. The multicolor LED color red would indicate that the wearer's body temperature is above normal.

The body temperature display device is designed to be sealed in a soft hypoallergenic and waterproof material such a silicone. The form factor of the body temperature display device is the size of a quarter and can be integrated with body worn apparel such as a hat or accessories such as a bracelet.

### BRIEF DESCRIPTION OF DRAWINGS

For a more complete understanding of the present disclosure and its advantages, reference is now made to the following detailed description, taken in conjunction with the accompanying drawings, in which:

FIG. 1 is the schematic of the body temperature display device.

FIG. 2 depicts side 1 of the body temperature display device typical form factor.

FIG. 3 is side 2 of a typical form factor of the body temperature display device.

FIG. 4 depicts the clear soft plastic waterproof body temperature display device cover

FIG. 5 depicts the body temperature display device integrated into a stretch fit bracelet.

FIG. 6 depicts the body temperature display device integrated into an adjustable fit bracelet.

FIG. 7 depicts the body temperature display device integrated into a face mask.

FIG. 8 depicts the body temperature display device integrated into a face mask while worn.

FIG. 9 depicts the body temperature display device integrated into a face mask while worn.

FIG. 10 depicts the body temperature display device integrated into a transdermal patch.

FIG. 11 depicts the body temperature display device integrated into a typical cap.

FIG. 12 depicts the body temperature display device integrated into a hardhat.

FIG. 13 is the flowchart of the functionality for the microcontroller.

### DETAILED DESCRIPTION OF THE INVENTION

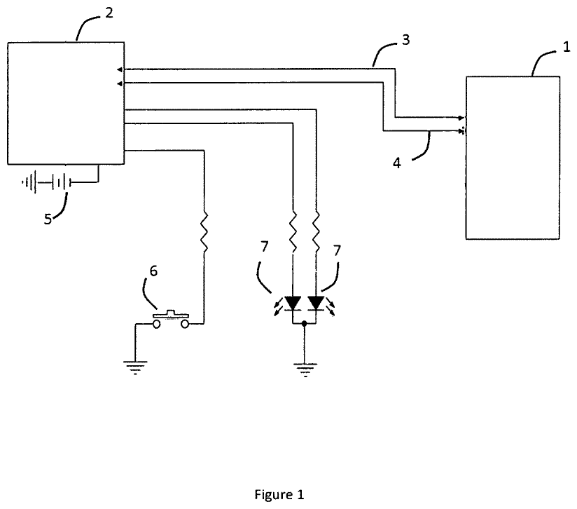

FIG. 1 depicts an exemplary block diagram of the body temperature display device 8. This figure depicts the use of a temperature IC (Integrated Circuit) 1 as known in the art, that when in close proximity to the exposed skin of the wearer, will read the wearer's body temperature to within a tenth of a degree accuracy. The outer surface of the temperature IC 1 is the sensor and can be extended beyond the surface of the temperature IC 1 by adhering a thermo conductive material to the surface of the temperature IC 1. When the body temperature display device 8 is integrated with body worn devices and apparel, the temperature IC 1 will be oriented to face the exposed skin of the wearer.

The light emitting temperature status multi-color LED 7 (Light Emitting Diode) as known in the art will be on the exterior face of the body temperature display device 8 opposing the temperature IC 1 so that is visible while body worn by the wearer. The multicolor LED 7 can be transitioned by the microcontroller 2 as is known in the art to be either Green, Red, or Orange. Flashing Orange will indicate initial power up and awaiting a valid body temperature of the wearer of the body temperature display device 8. Prolonged flashing orange would indicate the temperature is outside body temperature range or the temperature IC 1 is not close enough to the exposed skin of the wearer. When the body temperature of the wearer is normal, the LED 7 will be activated by the microcontroller 2 to transition to the color Green. When the body temperature of the wearer is above normal, the LED 7 will be activated by the microcontroller 2 to transition to the color Red.

The microcontroller 2 communicates commands to the temperature IC 1 and receives data from the temperature IC 1 using the command 3 and data 4 communication connections using serial or parallel communications as known in the art.

The body temperature display device is powered by a battery 5 which can be replaceable for a reusable body temperature display device 8 or fixed for a disposable body temperature display device 8.

The body temperature display device 8 is activated and deactivated by a switch button 6 that can be either momentary or alternate action as known in the art.

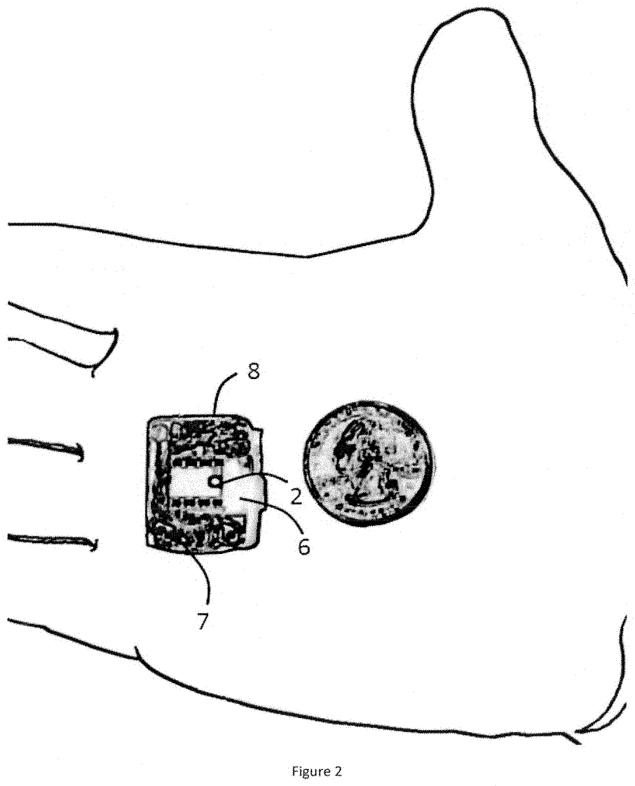

FIG. 2 depicts a typical physical form factor of the body temperature display device 8. The microcontroller 2 controls the body temperature display device. The multicolor LED 7 is located on the opposing side to the temperature IC 1 to be visible. The switch button 6 to activate and deactivate the body temperature display device is located on the side opposite the body facing side of the body temperature display device 8.

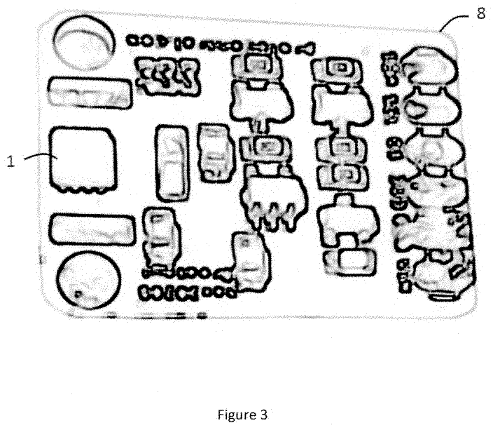

FIG. 3 depicts the opposite side of the body temperature display device 8 that faces the exposed skin. The temperature IC 1 is positioned as the component with the highest elevation on the body temperature display device 8 to be close to the exposed skin of the wearer of the body temperature display device 8.

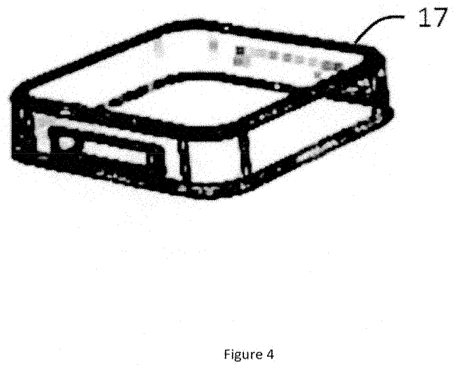

FIG. 4 depicts the clear soft waterproof plastic enclosure 17 that is sealed around the body temperature display device 8.

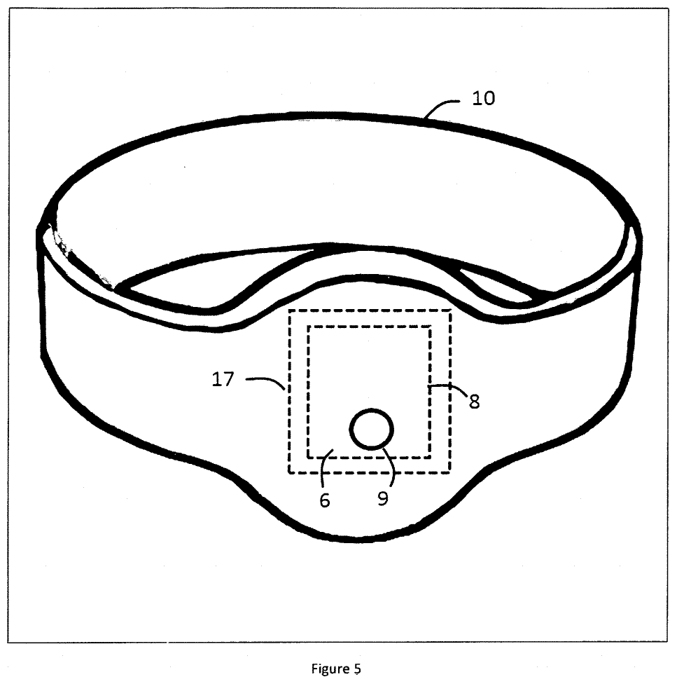

FIG. 5 depicts the body temperature display device 8 sealed in the clear soft waterproof plastic enclosure 17 further molded inside a soft flexible hypoallergenic bracelet 10. The multicolor LED 7 is exposed via a viewing hole 9. The body temperature display device 8 activate/deactivate switch button 6 (not visible) is transitioned by squeezing the soft flexible hypoallergenic bracelet 10 and the clear soft waterproof plastic enclosure 17 between the thumb and forefinger.

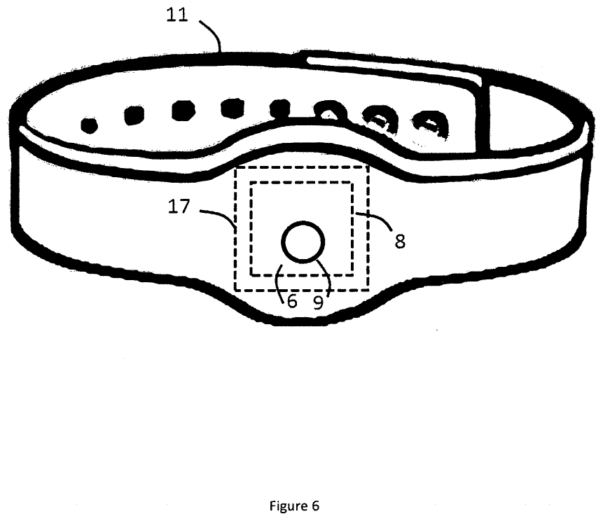

FIG. 6 depicts the body temperature display device 8 sealed in the clear soft waterproof plastic enclosure 17 further molded inside an adjustable soft flexible hypoallergenic bracelet 11. The multicolor LED 7 is exposed via a viewing hole 9. The body temperature display device 8 activate/deactivate switch button 6 (not visible) is transitioned by squeezing the adjustable soft flexible hypoallergenic bracelet 11 and the clear soft waterproof plastic enclosure 17 between the thumb and forefinger.

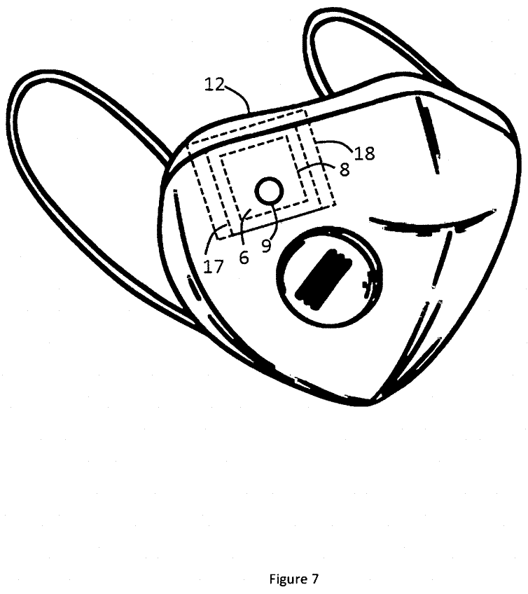

FIG. 7 depicts the body temperature display device 8 sealed in the clear soft waterproof plastic enclosure 17 further placed inside a sewn pocket 18 in a facemask 12. The multicolor LED 7 is exposed via a viewing hole 9. The body temperature display device 8 activate/deactivate switch button 6 (not visible) is transitioned by squeezing the clear soft waterproof plastic enclosure 17 between the thumb and forefinger.

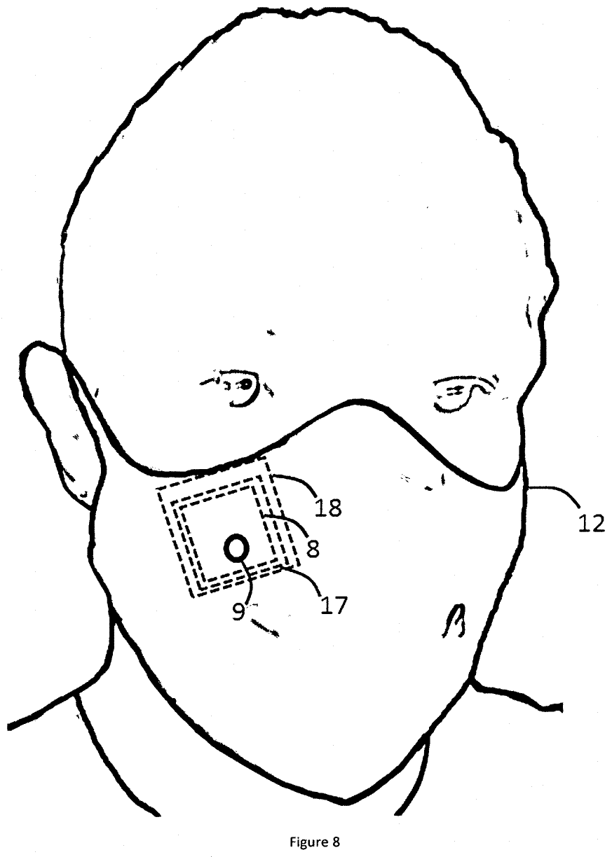

FIG. 8 depicts the body temperature display device 8 sealed in the clear soft waterproof plastic enclosure 17 further placed inside a sewn pocket 18 in a facemask 12 being worn.

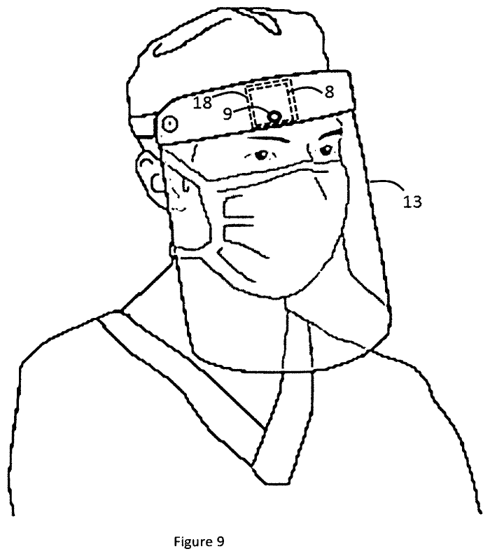

FIG. 9 depicts the body temperature display device 8 sealed in the clear soft waterproof plastic enclosure 17 further placed inside the headband of a face shield 13. The multicolor LED 7 is exposed via a viewing hole 9. The body temperature display device 8 activate/deactivate switch button 6 (not visible) is transitioned by squeezing the clear soft waterproof plastic enclosure 17 between the thumb and forefinger.

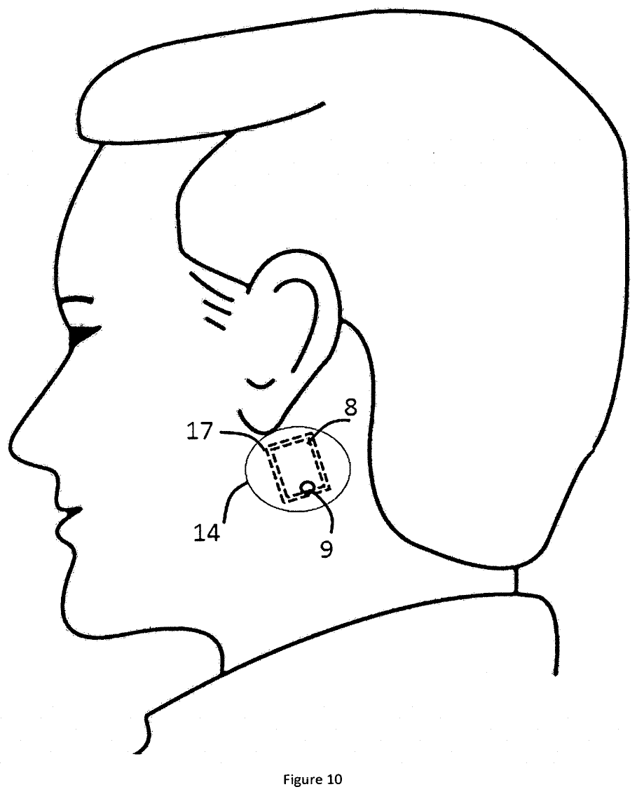

FIG. 10 depicts the body temperature display device 8 sealed in the clear soft waterproof plastic enclosure 17 further placed inside a transdermal patch 14. The multicolor LED 7 is exposed via a viewing hole 9. The body temperature display device 8 activate/deactivate switch button 6 (not visible) is transitioned by squeezing the clear soft waterproof plastic enclosure 17 between the thumb and forefinger.

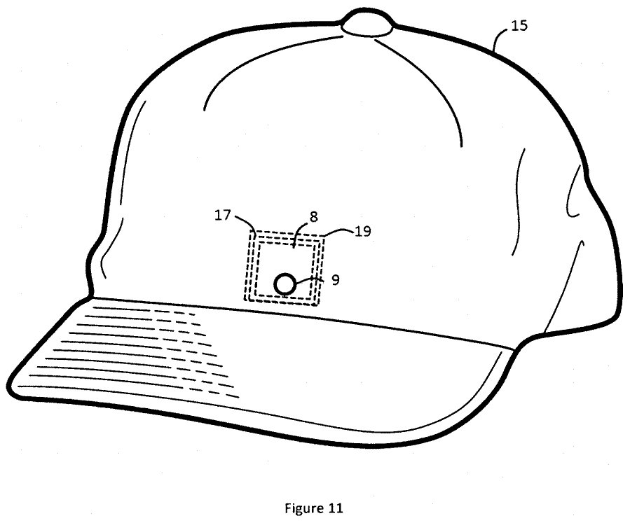

FIG. 11 depicts the body temperature display device 8 sealed in the clear soft waterproof plastic enclosure 17 further placed inside a sewn pocket 19 in a hat 15. The body temperature display device 8 activate/deactivate switch button 6 (not visible) is transitioned by squeezing the clear soft waterproof plastic enclosure 17 between the thumb and forefinger. The multicolor LED 7 is exposed via a viewing hole 9.

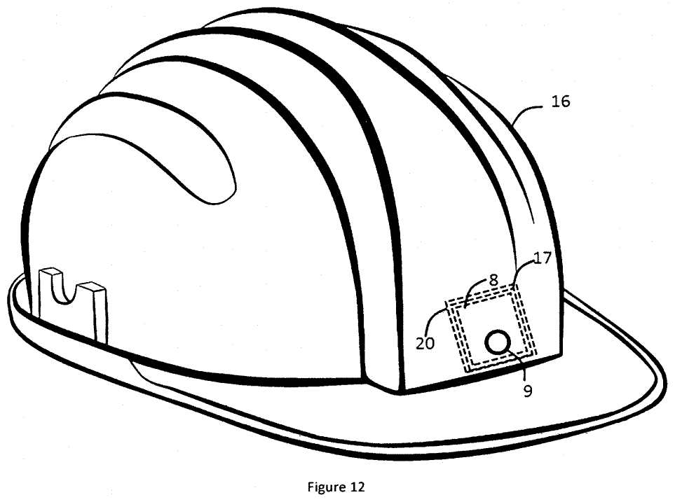

FIG. 12 depicts the body temperature display device 8 sealed in the clear soft waterproof plastic enclosure 17 further placed inside a pocket 20 inside the hard hat 16. The body temperature display device 8 activate/deactivate switch button 6 (not visible) is transitioned by squeezing the clear soft waterproof plastic enclosure 17 between the thumb and forefinger. The multicolor LED 7 is exposed via a viewing hole 9.

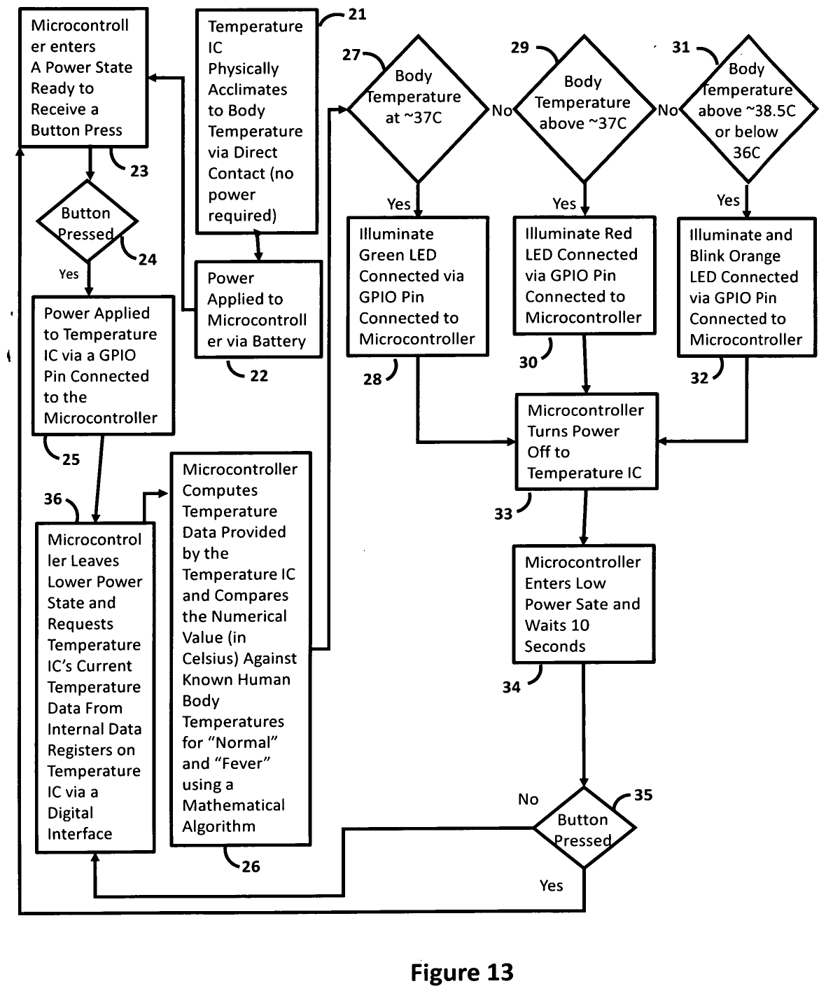

FIG. 13 is the flowchart for the body temperature display device 8 microcontroller 2 functionality. Once the body temperature display device 8 is placed against the exposed skin of the wearer the temperature IC acclimates to the wearer's body temperature. Once the battery 5 is attached 22, the microcontroller 2 is enabled to be activated 23 by the activate/deactivate switch button 6. Once the activate/deactivate switch button 6 is pressed 24 activation is applied 25 to the microcontroller 2. The microcontroller 2 then sends a command 36 requesting the temperature ICs current temperature data using the command connection 3 between the temperature IC 1 and microcontroller 2. The microcontroller 6 will receive temperature data from the temperature IC 1 using data connection 4 between the temperature IC 1 and the microcontroller 2. The microcontroller 2 will then compute the temperature data 26 provided by the temperature IC 1 comparing the computed temperature against a normal temperature 27, a fever temperature 29, or a temperature outside the human body range 31. If the temperature is within normal 28 the microcontroller 2 will illuminate the multicolor LED 7 to the color Green 28. If the temperature is above normal in the fever range 29 the microcontroller 2 will illuminate the multicolor LED 7 to the color Red 30. If the temperature is outside of the range of normal or fever 31 the microcontroller 2 will illuminate the multicolor LED 7 to the blinking color Orange 32. After activating the multicolor LED 7 to Green 28, Red 30 or blinking Orange 32, the microcontroller 2 will then turn power off 33 to the temperature IC 1. The microcontroller will enter a low power state 34 for 10 seconds. The microcontroller will then check 35 if the activate/deactivate switch button 6 has been pressed again if the activate/deactivate switch button 6 is momentary if the activate/deactivate switch button 6 is still closed if the activate/deactivate switch button 6 is alternate action. If the activate/deactivate switch button 6 has not been pressed again 35, then the microcontroller 2 will continue to remain in the powered state 36 and collect temperature data from the temperature IC 1. If the activate/deactivate switch button 6 has been pressed again 35, then the microcontroller 2 will enter a low power state 23 awaiting a activate/deactivate switch button 6 activation.

Any appropriate integration methods with other body worn items other than mentioned above may be used in accordance with the scope of the present disclosure.

What is claimed:

1. A body worn apparatus that displays the body temperature status of the wearer as either normal or fever.

2. The apparatus, according to claim 1 that displays body temperature status by means of a multicolor Light Emitting Diode (LED).

3. The apparatus, according to claim 1, that is powered by a replaceable or rechargeable battery.

4. The apparatus, according to claim 1, that is waterproof.

5. The apparatus, according to claim 1, that can be power cycled by squeezing the soft plastic enclosure.

6. The apparatus, according to claim 1, that displays when body temperature is not available.

7. The apparatus, according to claim 1, that can be placed inside a bracelet.

8. The apparatus, according to claim 1, that can be placed inside a facemask.

9. The apparatus, according to claim 1, that can be placed in a face shield.

10. The apparatus, according to claim 1, that can be placed in a transdermal patch.

11. The apparatus, according to claim 1, that can be placed in a hat.

12. The apparatus, according to claim 1, that can be placed in a hard hat.
